(19) United States
(12) Patent Application Publication
(10) Pub. No.: US 2006/0241689 A1
(43) Pub. Date: Oct. 26, 2006

(54) Viscera retainers, surgical drains and methods for using same

(76) Inventors: Arnold R. Leiboff, Stony Brook, NY (US); Joseph Zipper, MIller Place, NY (US)

Correspondence Address: BRIAN ROFFE, ESQ, 11 SUNRISE PLAZA, SUITE 303, VALLEY STREAM, NY, 11580-6170, US

(21) Appl. No.: 11/406,507

(22) Filed: Apr. 18, 2006

Related U.S. Application Data
(60) Provisional application No. 60/673,169, filed on Apr. 20, 2005.

(51) Int. Cl. A61B 17/08

(52) U.S. Cl. 606/213

(57) ABSTRACT

Viscera retainer which facilitates wound closure, for example, in the abdominal region, includes a body portion containing a tear seam. The body portion is inserted into the wound prior to closure to act as a physical barrier between the peritoneum and the viscera. A free extension of the body, or an attachment to the body, remains accessible outside the wound while a membranous layer of the abdominal wall is closed. When that closure is almost complete, the body portion is withdrawn from the abdomen by extracting it as a continuous ribbon through an opening. As the ribbon is withdrawn, the body tears along the tear seam generating free ribbon and shrinking the size of the residual body, until the entire body is removed from the abdominal cavity. The closure is then completed. A surgical drain has a planar body portion formed from a wound or wrapped tubular form having perforations in the body portion.

### CROSS-REFERENCE TO RELATED APPLICATION

This application claims priority under 35 U.S.C. § 119(e) of U.S. provisional patent application Ser. No. 60/673,169 filed Apr. 20, 2005, incorporated by reference herein.

### FIELD OF THE INVENTION

The present invention relates generally to surgical devices. In particular, the present invention concerns a viscera retainer, a surgical aid to facilitate closure of a wound following a surgical procedure. The viscera retainer is especially useful in abdominal surgery and it will be convenient to hereinafter describe the invention in relation to that exemplifying surgical application. It should be understood however that the viscera retainer in accordance with the invention is not limited to use in abdominal surgery and is capable of wider application and use.

The present invention also concerns a surgical drain, useful in, for example, draining body cavities following a surgical procedure.

### BACKGROUND OF THE INVENTION

Following surgery in the abdominal region, a wound, often of considerable size, needs to be closed by suturing. This is a delicate and time-consuming procedure as the surgeon must carefully suture the membranes of the abdominal wall back together while being very cautious not to nick or puncture the soft internal organs of the abdomen, known as the viscera. The procedure is made even more difficult if the patient is overweight because there is a greater need to retain, or hold down, the viscera while simultaneously pulling together the edges of the membrane being sutured, such as the peritoneum or fascia. In this situation, the risk of nicks or punctures to internal organs, such as the bowel, becomes even greater.

To alleviate this problem, surgeons have occasionally used a rectangular piece of rubber, or similar material, to retain the viscera and provide a physical barrier against accidental nicks or punctures. The rubber was inserted into the wound over the viscera prior to closure. The wound was then partially closed, by suturing together the edges of the peritoneum, over the rubber, until a hole of just sufficient size to remove the rubber remained. The rubber was then removed through that hole and the remainder of the wound closed. This procedure did, to some extent, address the problem but the stiffness of rubber and the awkward shape made it difficult to remove the piece of rubber from the wound through the hole.

One known device, sold under the name Glassman Viscera Retainer, the “Fish” by Adept-Med International Inc. partly addresses this problem. It includes a viscera retaining part, to be inserted into the wound, and a circular ring, to act as a handle to facilitate extraction of the retaining part from the wound. The retaining part includes a roughly diamond shaped, pliable web extending from a solid central rib. Attached to an end of the rib is a string, and at the end of the string is the circular ring. In use, the retaining part is inserted into the wound prior to closure and the wound is then partially closed by suturing over the retaining part. The retaining part is then withdrawn by pulling on the string using the ring. It has been found, however, that the solid central rib reduces the ability to remove the retaining part through a small hole.

Another product referred to as a “SurgiFish”, and sold by Greer Medical under U.S. Pat. No. 6,736,141, is purported to be easier to use and capable of being withdrawn through a smaller hole upon partial closure of the wound and can be re-used in subsequent surgical procedures.

Both these products are best used when a wound is closed from one end to the other. The products are more difficult to use when a surgeon closes the wound from both ends and completes his closure in the center. When the surgeon closes the wound in this manner, he often removes and replaces the product so that it can be removed through the center of the wound. This is an added inconvenience, which consumes times and effort.

Surgical drains to drain body cavities are well known and frequently used. Drains are frequently linear structures. They may be tubular, having a circular or rectangular profile, and are usually perforated in the portion that remains internally, within the body cavity, after surgery. An example of a rectangular drain with perforations in the portion that remains within the body is the Jackson Pratt drain. A squeeze bulb is attached the end of the non-perforated, circular external portion of the Jackson Pratt drain to apply gentle suction to withdraw fluid from the abdominal cavity. “Blake” drains, such as shown in U.S. Pat. No. Des. 288,962, have longitudinal undercut grooves in the portion that remains within the body cavity, instead of a tubular form. “Pigtail” drains are catheters that spiral in the perforated portion that remains within a body cavity, which aids in retention within the body cavity. However, each coil or spiral turn of these catheters remain separated, unattached, from the adjacent turn.

### OBJECTS AND SUMMARY OF THE INVENTION

It is an object of the present invention to provide a new and improved viscera retainer to facilitate wound closure.

It is another object of the present invention to provide a new and improved viscera retainer which enables a surgeon to close a wound from both ends and complete the closure in the center while allowing easy removal fo the viscera retainer through a hole in the center of the wound.

It is yet another object of the present invention to provide a new and improved surgical drain for use in, for example, draining body cavities following a surgical procedure.

In order to achieve these objects and others, a viscera retainer in accordance with the invention comprises a generally planar body incorporating a tear seam. The body is made predominantly of a flexible and resiliently stretchable material, for example, surgical grade silicone or PVC. The body may be entirely removed from a cavity or lumen, after the wall of the cavity or lumen has been nearly completely closed, by pulling on an end thereof. As a portion of the body is drawn out through a small opening in the wall, the body tears along the tear seam, transforming the body into a continuous strip that can be easily withdrawn through the small opening in the wall.

A first embodiment comprises a ribbon wound as a spiral in a single plane to form a planar body. The ribbon has an exaggerated obround (racetrack)cross-section. The ribbon is fixed in a spiral formation either by heat welding or solvent welding or by some other process that joins the edges of adjacent convolutions or turns of the spiral to one another. The spiral joint formed thereby serves as a tear seam along which the spiral separates as the body unwinds when withdrawn from the cavity or lumen. Alternatively, the body may be formed by a molding process and the tear seam formed either in the molding process by creating a spiral of thinned material, or after the molding process forming a spiral of perforations by pressing the teeth of a spiral toothed blade through the body. Near the center of the spiral, the end of the ribbon remains free, unattached to any adjacent convulution, and may extend for a length outside the plane of the spiral.

The viscera retainer described above is especially useful to facilitate closing of a wound following abdominal surgery. In use, the body is inserted into the wound prior to closure, to act as a physical barrier protecting the viscera. The inner free end of ribbon is not inserted into the wound, and remains accessible to facilitate later withdrawal of the entire body from the wound. If the wound is to be closed from both ends toward the middle, the inner free end is brought out of the wound at the center of the wound. If the wound is to be closed from one end to the other, a free end of ribbon is created at the perimeter by tearing ribbon from body, and is brought out at the end of the wound to be closed last. The inner free end, if present, may be excised. A membranous layer of the abdominal wall (peritoneum or fascia) is then drawn together and sutured over the body of the viscera retainer. All sutures may be placed. The body portion is then withdrawn from the wound by pulling on the accessible free end. As the free end is pulled, the ribbon is progressively torn from the body at the tear seam and the body unwinds as it is withdrawn. The entire body can thus be removed through a small opening in the membranous layer being closed.

A rigid extraction probe containing a loop with a narrow obround aperture facilitates removal of the device from the abdomen after closure of the peritoneal or fascial membranes is nearly complete. The accessible end of the ribbon is passed through the loop, and the loop is slid over the ribbon through the wound to a point just beneath the membrane being closed. To withdraw the body, the ribbon is pulled through the loop, which facilitates separation of the adjacent convolutions of the ribbon along the tear seam.

The optimum size of the viscera retainer will depend on the size of the wound being closed. However, this device has the advantage that it can easily be reduced in size by unwinding convolutions of the spiral and excising the unwound parts of the ribbon at the body's perimeter.

Fabricating the body from silicone or polyvinyl chloride (PVC) is also advantageous because the surfaces of these materials, when in contact with body fluids present during surgery, slide easily with respect to the organs and tissues of the patient. This further facilitates withdrawal of the viscera retainer from the wound through a small opening.

Impregnating the device with barium sulphate to render it radiopaque is also advantageous. In this way, if by any chance a segment or fragment of the viscera retainer remained within the abdomen of the patient after the wound had been closed, it would be detectable in an X-ray.

Other embodiments include those where the width of the ribbon varies along its length. Having the ribbon narrow near the center of the spiral has the advantage of permitting a smaller aperture at the center of the spiral, where a wider ribbon could not assume a small radius of curvature. When the ribbon widens towards the periphery of the body, a shorter overall length of ribbon can be provided for a given body diameter. Having the most peripheral segment of the ribbon narrow may have an advantage when closing a wound from one end to the other and arranging for withdrawal of the body from its periphery. In this case, the peripheral end of the ribbon remains outside the wound and accessible while the wound is closed, and functions as a handle to initiate withdrawal of the body. A narrower, more supple handle interferes less with wound closure. The loop of the extraction probe for a device having a ribbon of variable width, would be just wide enough to accommodate the maximum ribbon width.

Another embodiment of a viscera retainer in accordance with the invention replaces the ribbon, which has an obround cross-section, with a cord, which has a circular cross-section. The corresponding extraction probe would have a circular loop. The cord may have a constant diameter, or its diameter may vary, in a similar manner and for a similar purpose that the width of the ribbon varies in the embodiment described above. Other cross-sectional shapes, such as rectangular or elliptical, can be used instead of obround or circular, combined with extraction probes having loops of corresponding shape.

Rather than having solid profiles, the coiled or wrapped structure can have tubular profiles. For example, instead of a circular cord wound into a planar structure, a tube can be wound or coiled into a planar structure. This may have the advantage of saving on material. The tubular structure may be perforated with multiple holes. A planar coil of such a tube could then have an additional function other than a visceral retainer, namely as a drain. Such a device could be left in the abdominal cavity with its tail end exiting either through the incision, or through a separate stab wound in the abdominal wall. In the postoperative period, vacuum pressure could be applied to the tail end of the device extending out of the body, and the device would function as a drain, withdrawing fluids from the abdominal cavity. This could be particularly useful if those fluids are contaminated or infected. When no longer needed, this device could then be removed from the body merely by pulling on the tail end, and slowly drawing it out (as the tube uncoils or unwinds). If the tube is provided with multiple perforations (with the exception of the tail end which is not perforated and is operatively positioned to be the only part extending out of the body), vacuum applied to the tail end would not only draw fluid through the tube, but also across the walls of the tube. For example, if the tube were circular with multiple perforations, and was wound in a coil, then suction applied at the non-perforated tail end emerging from the inner aspect of the coil would not only draw fluids into the tube through the perforations and then along the axis of the tube, but would also move fluid from the periphery of the coil to the center of the coil by fluid exiting the tube through a perforation at a more peripheral coil and re-entering the tube through an adjacent perforation of the adjacent more central coil.

Another aspect of the present invention provides a method of closing a wound following a surgical procedure. The method includes providing a viscera retainer of a type described above and inserting the body portion of the viscera retainer into the wound to act as a physical barrier between underlying tissue and the membrane to be closed. An end of the ribbon which forms the body is retained outside the wound and remains accessible. The membrane is almost completely closed and the body portion is withdrawn by pulling on the exterior end of the ribbon. The body tears along the tear seem as it is extracted. Closure of the wound is then completed.

A further aspect of the present invention provides a method of closing a wound following abdominal surgery. The method includes providing a viscera retainer of the type described above and inserting the body portion of the viscera retainer into the wound to act as a physical barrier between the abdominal wall and the viscera. A free end of the ribbon which forms the body is placed outside the wound and remains accessible. The peritoneum or fascia are then partially closed by suturing over the body portion and when the wound is almost completely closed, the body portion is withdrawn by pulling on the accessible end. Closure of the wound is then completed.

A still further aspect of the present invention is the use of a probe as described above to facilitate extraction of the body. When the wound is almost completely closed, the accessible end of ribbon is passed through the loop of the probe. The loop of the probe is passed over the ribbon and introduced into the wound beneath the closed membrane and the body is extruded through the loop, which facilitates separation of adjacent convolutions of ribbon form the body along the spiral tear seam. When the body is completely removed, the wound is completely closed.

It will be convenient to hereinafter describe the invention by reference to the accompanying drawings which illustrate a preferred embodiment thereof. Other embodiments of the invention are possible, and consequently the particularity of the accompanying drawings is not to be understood as superceding the generality of the preceding description of the invention.

### BRIEF DESCRIPTION OF THE DRAWINGS

The invention, together with further objects and advantages thereof, may best be understood by reference to the following description taken in conjunction with the accompanying drawings wherein like reference numerals identify like elements.

FIG. 1 illustrates a top view of a first embodiment of a viscera retainer in accordance with the invention.

FIG. 2 illustrates a cross-sectional view of the viscera retainer shown in FIG. 1 through the plane marked A-A.

FIG. 3 illustrates a probe that is useful in aiding withdrawal of the viscera retainer illustrated in FIGS. 1 and 2 from a cavity through a small opening left in the wound.

FIG. 4 illustrates the viscera retainer shown in FIG. 1 in use wherein the viscera retainer has been inserted into the abdominal cavity through an abdominal wall wound prior to closure;

FIG. 5 illustrates the wound nearly completely closed and the viscera retainer illustrated in FIG. 1 being drawn out of the abdominal cavity with the aid of the probe shown in FIG. 5.

FIG. 6 illustrates a perspective view of a second embodiment of a viscera retainer in accordance with the invention

FIG. 7 illustrates a top view of a third embodiment of a viscera retainer in accordance with the invention.

FIG. 8 illustrates a perspective view of a fourth embodiment of a viscera retainer in accordance with the invention.

FIG. 9 illustrates a perspective view of a fifth embodiment of a viscera retainer in accordance with the invention.

FIG. 10 illustrates a segment of a perforated tube that may be coiled into a viscera retainer or abdominal drain.

FIG. 11 illustrates a cross-sectional view of a sixth embodiment of a viscera retainer, which can also be used as an abdominal drain.

### DETAILED DESCRIPTION OF THE INVENTION

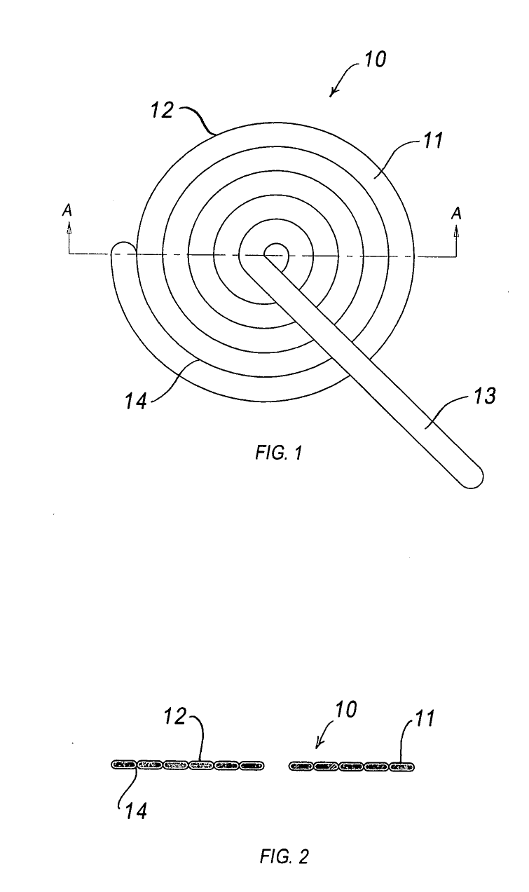

Referring to the accompanying drawings wherein like reference numerals refer to the same or similar elements, with reference to FIGS. 1 and 2, there is shown a viscera retainer 10 in accordance with the invention for facilitating wound closure following surgery, for example, in the abdominal region of a patient. Viscera retainer 10 includes a body portion 12 which is substantially round and may be formed as a spiral from a continuous ribbon 11 of an elastomeric material such as, but not limited to, silicone or PVC. The ribbon 11 has an obround cross-section (see FIG. 2). An inner end 13 of the ribbon 11 is free and extends outside the plane of the body portion 12. The convolutions of the ribbon 11 are fixed to adjacent convolutions along a spiral joint. The spiral joint defines a tear seam 14 along which the convolutions of ribbon 11 separate as they are pulled away from a remaining portion of the body portion 12, which occurs, for example, as the body portion 12 is extracted from the abdominal cavity through a small opening in the abdominal wall wound.

To ensure that fragments of the viscera retainer 10 are not left undetected within the abdomen of the patient after the wound has been closed, the elastomeric material may be impregnated with a material such as barium sulphate to render it radiopaque. In this manner, if a fragment of the viscera retainer 10 were left within the body after closure of the wound, it would be detectable using a conventional X-ray. A final step in a wound closure procedure using such a radiopaque viscera retainer would thus be to take an X-ray of the wound to ascertain whether any portion of the viscera retainer 10 remains in the body.

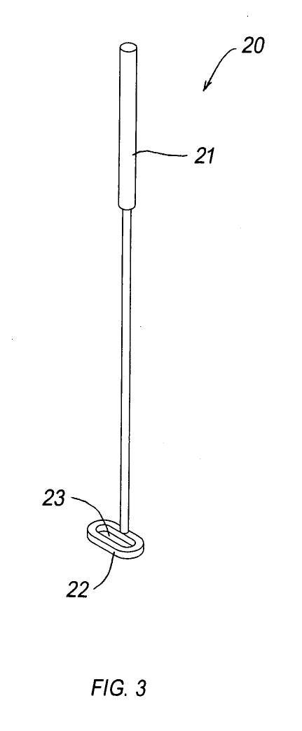

FIG. 3 shows an extraction probe 20 with a handle 21 and a ring or loop 22 defining an obround aperture 23 of a size just large enough to accommodate ribbon 11. The probe 20 is rigid and may be made out of metal or plastic.

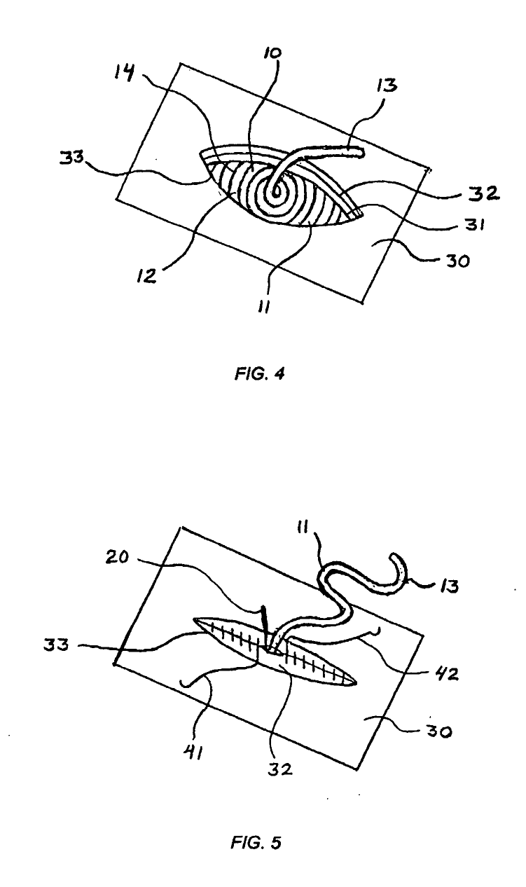

FIGS. 4 and 5 show the viscera retainer 10 in use. As shown in FIG. 4, the body portion 12 of the viscera retainer 10 is introduced into an abdominal wound 33 before its closure. Viscera retainer 10 acts as a physical barrier between the peritoneum 31 and the viscera. The inner free end 13 of the ribbon 11 which forms the viscera retainer 10 remains accessible outside the wound 33.

The peritoneal layer 31 or fascial layer 32 of the abdominal wall 30 is closed by suturing over the body portion 12 of the viscera retainer 10 starting from the ends of the wound 33 with two separate sutures 41 and 42 and sewing with each toward the middle. When closure of the fascia 32 is almost complete (FIG. 5), the body portion 12 is extracted from the wound 33 by pulling first on the free end 13 of ribbon 11 and then withdrawing the entire ribbon 11 comprising body portion 12. During withdrawal of the ribbon 11, the ribbon 11 unwinds, i.e., the convolutions of the ribbon 11 separate from one another along tear seam 14 to form a long strip which passes through the small opening remaining the fascia 32.

Alternatively, in order to assist in the extraction of ribbon 11, probe 20 is utilized. To this end, free end 13 of ribbon 11 is passed through aperture 23 of loop 22 of probe 20. Loop 22 is then slit over ribbon 11 through wound 33. Ribbon 11 is then pulled up through loop 22. Loop 22 facilitates separation of adjacent convolutions of ribbon 11 along tear seam 14.

After removal of the entire body portion 12, closure of the fascia 32 is completed by placing additional sutures, if necessary, or by pulling taught on sutures already placed, and tying the suture ends to one another.

The inner free end 13 of the ribbon 11 may optionally be provided with an aperture to enable a surgeon to insert his finger or forceps into the aperture to facilitate removal of the body portion 12 from the wound. If such an aperture is provided, the area around the aperture may be widened to ensure the inner free end does not tear when pulled by the surgeon.

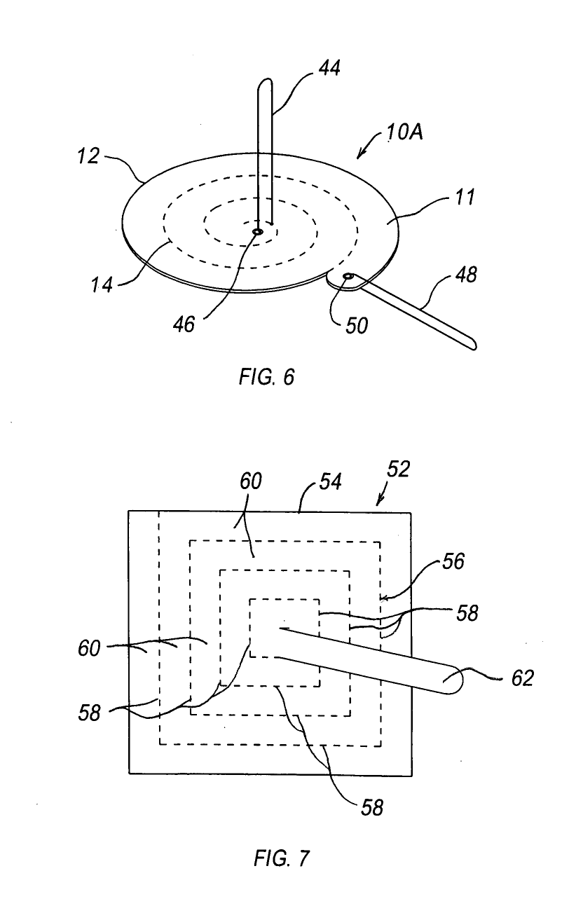

With reference to FIG. 6 which shows a viscera retainer 10A similar to viscera retainer 10, instead of providing a length of the ribbon 11 as the inner free end 13 to enable removal of the body portion 12 from the wound, it is possible to attach a loop of material 44 to the inner end of the ribbon 11 which lies in the plane of the body portion 12, e.g., pass the loop of material 44 through a small aperture 46 formed in the body portion 12 at or proximate the innermost part of the body portion 12 and in the innermost convolution of the body portion 12. Part of the loop of material 44 is designed to be maintained outside of the wound so that it can be grasped by the surgeon when it is desired to remove the body portion 12 from the wound. Pulling on the loop of material 44 will cause the ribbon 11 to separate along the tear seam 14. A similar loop of material 48 could alternatively or additionally be attached to the outer free end of the ribbon 11, e.g., by passing it through an aperture 50, to enable the possibility of removal of the body portion 12 from either end.

Although viscera retainers 10, 10A each include body portion 12 shaped as a spiral, viscera retainers in accordance with the invention can be constructed having other shapes. For example, with reference to FIG. 7, a viscera retainer 52 in accordance with the invention may have a substantially rectangular or square body portion 54 with a tear seam 56 formed as a series of linear, interconnected segments 58 defining elongate strips 60 therebetween. The tear seam 56 allows the body portion 54 to separate into a ribbon of elongate strips 60 when an inner free end 62 of the viscera retainer 52 is pulled.

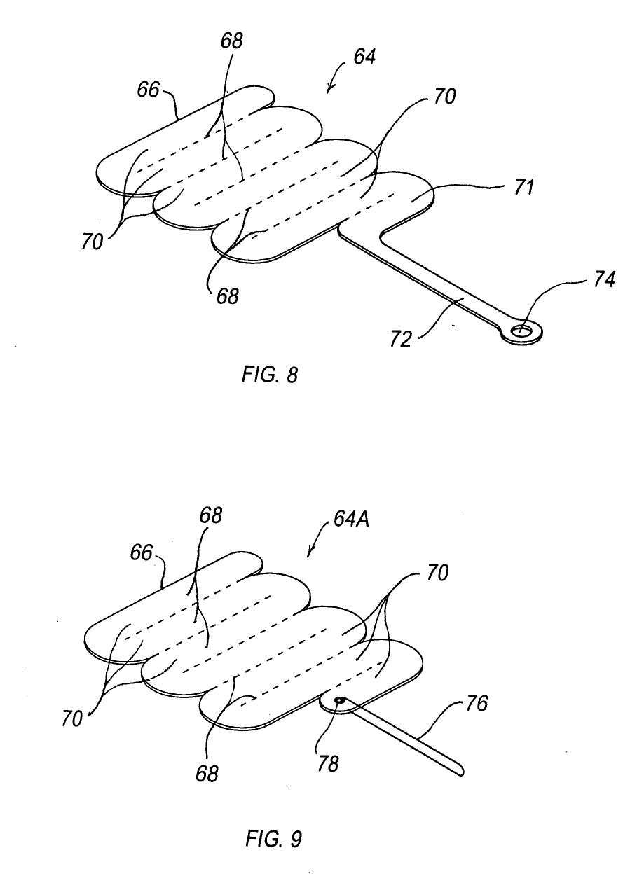

With reference to FIGS. 8 and 9, another alternative shape of a body portion 66 of a viscera retainer 64, 64A in accordance with the invention is a generally rectangular shape wherein, instead of a series of linear, interconnected segments forming a tear seam as described immediately above, a series of individual, parallel tear seams 68 are provided to give the body portion 66 a serpentine form. Alternating ones of the tear seams 68 extend inward from opposite sides of the body portion 66. The tear seams 68 define elongate sections 70 of the body portion 66, and the end of the sections 70 may be rounded.

As shown in FIG. 8, a free end portion 72 of the viscera retainer 64 is connected to or extends from an outermost one of the sections 70, for example, from the medial end of the section 71 as shown. When the surgeon pulls the free end portion 72 during the wound closure procedure, the sections 70, 71 separate from one another along the tear seams 68 to form an elongate ribbon.

The free end portion 72 may be provided with an aperture 74 to enable a surgeon to insert his finger or forceps into the aperture 74 to facilitate removal of the body portion 66 from the wound. If such an aperture 74 is provided, the area around the aperture 74 may be widened as shown to ensure the free end portion 72 does not tear when pulled by the surgeon.

As shown in FIG. 9, in viscera retainer 64A, a loop of material 76 is attached to an outermost one of the sections 70 by passing it through an aperture 78 therein. Viscera retainer 64A is thus not provided with a free end portion 72 as shown in FIG. 8. The surgeon can then remove the viscera retainer 64A by pulling on the loop of material 76.

The viscera retainers of the present invention thus make wound closure faster and easier, and reduces the risk of needle stick and needle perforation injury, to both the viscera of the patient and to the surgeon.

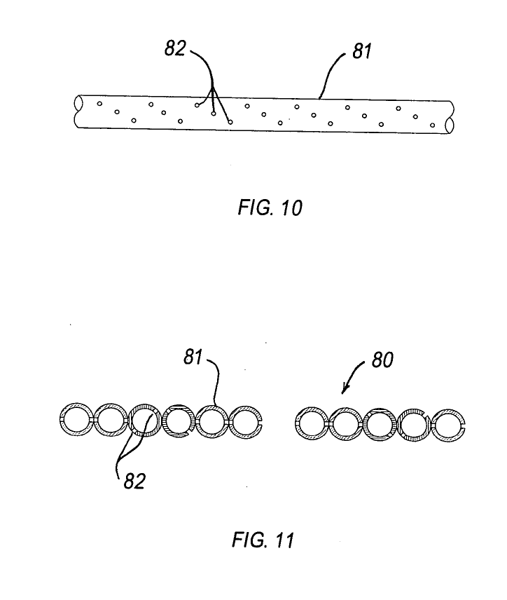

Referring now to FIGS. 10 and 11, a device 80 which functions as a viscera retainer may be constructed from a round tube 81, which is wound into a coil and sealed into the coil by heat, or adhesive or solvent welding. Device 80 may be made of plastic. A top view of device 80 could be similar to the top view of the first embodiment as shown in FIG. 1. In use in the abdominal cavity, the inner or outer end of the coil can be left long as a tail (i.e., a tail end), unattached to the adjacent coil, which can then be operatively brought or maintained outside the abdomen, as a handle to withdraw the device 80 through a small defect between stitches in a wound when the fascial closure is nearly complete, or whenever otherwise desired.

Tube 81 which forms the device 80 can have multiple perforations 82, as shown in FIG. 10. When tube 81 is wound into a coil, perforations 82 allow the interior of the tube 81 to communicate with the exterior of the tube 81, so that the device 80 could function as a drain, in addition to a viscera retainer. Left inside the abdomen after abdominal wound closure, the tail end of the tube 81 which had been exteriorized through the wound or through a separate stab wound in the abdominal wall, could be connected to a suction source. Fluid in the abdomen can then pass into the lumen of the device 80, i.e., the interior channel defined by the tube 81, and would be suctioned through the tube 81 and out through the tail end of the tube 81 that passes through the abdominal wall. Either end, or both ends of the coiled device 80 could be brought out through the abdominal wall.

Since some perforations of adjacent coils would line up with perforations of an adjacent coil, as seen in FIG. 11, fluid suctioned through the device 80 could travel not only in a spiral direction by staying within the lumen, but also in a radial direction, as the fluid may pass directly from one coil to the other through the aligned perforations. When the device 80 is no longer needed, it may be withdrawn from the abdominal cavity by pulling on the tail end that is situated exterior of the abdomen.

Other shapes of the drain, for examples rectangular shapes, where a perforated tube oscillates rather than spirals to form the wider planar form, are also included in this invention.

Although a preferred embodiment of the invention has been described herein in detail, it will be understood by those skilled in the art that variations may be made thereto without departing from the spirit of the invention. For example, although mention is made of the use of the devices described above for abdominal surgeries, it will be understood by one skilled in the art of medical devices that the same devices can be used for other purposes not associated with abdominal surgery.

1. A device for use in a surgical procedure, comprising:
a body having a planar form; and
means incorporated into said body for transforming said body from its planar form into a narrower, elongate form.

2. The device of claim 1, wherein said means comprise at least one seam arranged to tear to cause the transformation of said body.

3. The device of claim 2, wherein said at least one seam is a single spiral seam.

4. The device of claim 2, wherein said at least one seam comprises a plurality of seams arranged to provide said body with a serpentine, oscillating form when in its planar form.

5. The device of claim 1, wherein said body is structured and arranged for use as a viscera retainer useful when closing a body cavity whereby upon placement of said body in the cavity prior to surgical closure of the cavity, with an extension passing outward through an opening in the cavity wall, the extension is pullable after closure of the cavity causing transformation of said body from its planar form into its narrower, elongate form as said body is withdrawn through the opening in the cavity wall.

6. The device of claim 1, wherein said body is made of an elastomeric material.

7. The device of claim 1, wherein said body is made of rubber, polyvinyl chloride, polyurethane or silicone.

8. The device of claim 1, wherein said body comprises an elongate tube having a tubular wall defining a central lumen.

9. The device of claim 8, wherein said body further comprises perforations in said tubular wall.

10. The device of claim 8, wherein said elongate tube is arranged in a coil to form said planar body.

11. The device of claim 8, wherein said elongate tube has an oscillating arrangement to form said planar body.

12. The device of claim 8, wherein said body is structured and arranged for use as a surgical drain to drain a body cavity.

13. A method for retaining viscera during closure of a wound in a body cavity wall, comprising:
providing a body having a wide planar form and capable of being transformed into a narrower, elongate form;
placing the body when having its wider planar form beneath the wound between the viscera and the body cavity wall; then
only partially closing the wound with the body overlying and protecting the viscera; then
removing the body by pulling it out of the cavity through a remaining space in the partially closed wound; and
constructing the body such that pulling on an end thereof transforms the body from its wider planar form into its narrower, elongate form that fits through the remaining space in the wound.

14. The method of claim 13, further comprising completing the closure of the wound once the body has been removed.

15. The method of claim 13, wherein the step of providing a body having a wide planar form and capable of being transformed into a narrower, elongate form comprises forming a seam in the body.

16. A method for draining a body cavity, comprising:
providing a body having a wide planar form and a free end portion and capable of being transformed into a narrower, elongate form defining a central lumen and perforations;
placing the body having its wider planar form in the body cavity;
positioning the free end portion out of the body cavity through an opening in a wall of the body cavity,
after closure of the body cavity, allowing fluid from the body cavity to drain into the central lumen and out of the body cavity via the free end portion,
when drainage is no longer needed, removing the body from the body cavity by pulling the free end portion, and
constructing the body such that upon pulling the free end portion, the body transforms from its wider planar form into its narrower, elongate form and fits through the opening in the body cavity wall.

17. The method of claim 16, wherein the step of providing a body having a wide planar form and capable of being transformed into a narrower, elongate form comprises forming a seam in the body.

18. A method for removing a body having a wider planar form which is transformable into an elongate, narrower form from a body cavity through a narrow opening in the cavity wall, comprising:
positioning a free end of the body exterior of the body cavity,
positioning the free end through a ring having similar shape and dimensions as the
circumference of body when having its elongate, narrower form, and pulling the body through the loop so that an edge of the loop facilitates transformation of the body from its wider planar form into its elongate, narrower form such that the body is removable from the body cavity through the narrow opening.

19. The method of claim 18, further comprising forming a seam in the body to enable its transformation upon pulling through the loop.
